(19) United States
(12) Patent Application Publication
(10) Pub. No.: US 2008/0260639 A1
(43) Pub. Date: Oct. 23, 2008

(54) PREPARATION AND METHOD UTILIZING RADIOLABELED CHLOROTOXIN

(76) Inventors: Robert G. McKenzie, Tampa, FL (US); Booke Schumm, Ellicott City, MD (US)

Correspondence Address: BROOKE SCHUMM III;Daneker, McIntire, Schumm, Prince, Goldstein et al, ONE NORTH CHARLES STREET, SUITE 2450, BALTIMORE, MD, 21201, US

(21) Appl. No.: 12/024,087

(22) Filed: Jan. 31, 2008

Related U.S. Application Data
(63) Continuation-in-part of application No. 11/611,862.
(60) Provisional application No. 60/943,297, filed on Jun. 11, 2007.
(60) Provisional application No. 60/580,455, filed on Jun. 17, 2004.

(51) Int. Cl. A61K 51/08; A61K 51/04

(52) U.S. Cl. 424/1.69; 424/1.65; 424/1.85

(57) ABSTRACT

The inventors propose to label a chlorotoxin with I-123 or I-124 for a diagnostic agent, and with I-124 or I-131 for a therapeutic agent to reduce tumors and then to use a process they previously invented to stabilize the radiopharmaceutical. The inventors propose to reconstitute the preferred agents and administer them cyclically to diagnose and treat tumors. The inventors also propose a TETA or DOTA link to a metal radioisotope.

This application claims benefit of a provisional application of the same name filed on Jun. 11, 2007 as U.S. Appl. 60/943,297 and to the extent required, is a continuation in part of that application, and is a continuation in part of U.S. application Ser. No. 11/611,862 which in turn is a national stage entry from PCTUS05/21847, which claims benefit of provisional applications including U.S. Provisional Application 60/580,455 filed Jun. 17, 2004.

### FIELD OF INVENTION

This invention relates to the medical arts, in particular to radiopharmaceutical diagnostic and therapeutic agents.

### SUMMARY OF INVENTION

The inventors propose to prepare a radiopharmaceutical utilizing a chlorotoxin, or a synthetic equivalent to a chlorotoxin by a novel method for surgical implant or needle injection or IV for treatment of tumors, especially tumors refractory to other treatment, which are reduced by uptake of chlorotoxin. The inventors propose to use a different radioactive tag than the literature proposes which is enabled by the unique method described herein. The inventors propose to use I-123 labeled chlorotoxin lyophilized according to this invention for diagnostic imaging.

The inventors propose to label chlorotoxin and stabilize it to prevent hydrolysis and autoradiolysis by a method of lyophilization described herein which will enable higher purity and higher concentrations of radioisotope for treatment. The preferred mode is using I-123 labeled chlorotoxin for diagnostic use, and I-124 for treatment because it has a shorter half-life than I-131, but I-123 and I-131 can be used cyclically as well.

### BACKGROUND

The lead candidates proposed for the invention are peptides from a scorpion. Generally, they are referred to as Chlorotoxins. In particular, chlorotoxin is intended to include RBmK CTa, as described in Fu Y J, “Therapeutical Potential Of Chlorotoxin-Like Neurotoxin From The Chinese Scorpion For Human Gliomas,” 412 (1) Neuroscience Letters 62-67 (2007 Jan. 22) (electronic publ. Dec. 12, 2006) derived from the venom of scorpion Buthus martensii Karsch (see also Wu et al (38 Toxicon 661-668 (2000)), and TM-601™ described in Mamelak A N, “Phase I Single-Dose Study Of Intracavitary administered Iodine 131-TM-601 In Adults With Recurrent High-Grade Glioma,” 24(22) J. Clinical Oncology 3644-3650 (Aug. 1, 2006), I-125 labeled CTX derived from a venom of the Giant Yellow Israeli scorpion, Leiurus quinquestriatus, “Use Of Chlorotoxin For Targeting Of Primary Brain Tumors,” 58(21) Cancer Research 4871-4879 (Nov. 1, 1998), and also described in 39(2) Glia 162-73, (August 2002) (collectively all such compounds referred to as “chlorotoxin”). TM-601 is a trademark of TransMolecular, Inc.

As often occurs, the difficulty is to image the cancer accurately, and as well, to find a suitable tumor targeting agent to degrade the tumor radioactively while minimizing damage to surrounding tissue.

Moreover, as cancer treatment becomes more nuanced, it is important to be able to have standby supply of product which does not degrade. Peptides such as chlorotoxin are complex in shape, and are susceptible to damage from radiation from radiolysis, which usually generates free oxygen or free hydroxyl radicals, which attack the peptides and alter their properties. Absent this invention which eliminates water during storage, radiotagged chlorotoxins are susceptible to oxidation damage from hydroxyl compounds created by the impact upon shell electrons of water and their dislodgement by radioactive bombardment. I-131 has a half-life of 8 days, which has several disadvantages, the most notable of which is that a high dose will not decay quickly causing adjacent healthy tissue to be damaged, and at a high dose, causing radiolysis unless the radiopharmaceutical is radio-tagged immediately prior to administration. If a patient does not appear on time, or if a hospital does not have a nuclear pharmacy, then the radiolabeled ligand must be disposed.

I-124 was suggested as a PET imaging agent with chlorotoxin, but is not suggested as a therapeutic agent, in part because absent the stabilization and lyophilization of this invention, the intense radioactivity of the I-124 needed is deleterious to the effect of the ligand.

In contrast to the Wolfangel, “Stabilized Therapeutic Radiopharmaceutical Complexes, U.S. Pat. No. 5,219,556, Jun. 15, 1993 invention which stated: “the lyophilization step itself generally takes about 24 hours to perform,” the present invention proposes to produce a stable radiopharmaceutical complex by a lyophilization process which “freeze-dries” the complex in five hours or less, normally 2-4 hours, and then requires no further refrigeration. A recent text by Saha on Nuclear Pharmacology refers to the necessity of a heating step; this invention eliminates that time-consuming and potentially deleterious step.

### OBJECTIVES OF THE INVENTION

It is an objective to develop a new effective combination of preparation and selection of radiopharmaceutical agent to a storable reconstitutable form to more effectively attack tumors, particularly brain tumors which are more intractable because of blood brain barrier issues.

### DESCRIPTION OF INVENTION

For imaging, this invention is enabled by the interaction of the methodology of eliminating water, and by using a shorter half-life gamma emitter, namely I-123, and using it to radiolabel the chlorotoxin. The advantages are that there is a rapid decay of the radioactive substance, and minimization of the effects on the patient, and the product can be shipped and stored. Even with overnight shipment, by using the rapid lyophilization technique described herein with I-123, an increased amount of half-lives are available to enable the shipment and storage, and even after the passage of two half-lives, the product would be of sufficient purity to use. The complaint about short-half-life compounds is often that the radiolabeling has to take place at such high concentrations that by the time the product arrives, either there is no radioactivity, or because of the initial higher concentration, the ligand is destroyed.

While literature discusses I-131 extensively, and for on-site radiolabeling, it has advantages of high energy emissions, those emissions make it less desirable for shipment and storage unless this invention is applied to lyophilize and stabilize the material. Not every location has a nuclear pharmacy and so remote shipping is important.

Because I-123 is a lower energy than I-131, to achieve a similar effect for tumor destruction, a higher amount of ligand that is radiolabeled could be used to achieve similar effect to the I131. The literature suggests that dose-limiting toxicities were not observed.

I-131 has a 606.3 KeV β⁻ emission (89%) and 364 KeV (81.4%) gamma emission with the 8.02 day half-life. By using roughly three times the radiolabeled ligand, higher emissions could be achieved by the I-123. The difference in energy illustrates why I-123 chlorotoxin would be a suitable IV administered short half-life diagnostic.

For tumor targeting, in order to achieve a more desirable half-life characteristic than I-131, but achieve a similar energy, the inventors propose to use I-124 chlorotoxin. I-124 has a 4.18 day half-life, but achieves significantly higher energies than I-123 for tumor therapy, comparable to I-131. I-124 has the advantage of decaying faster enabling more treatments and diagnosis cycles than I-131. I-125 could be utilized.

While literature has generally mentioned iodine isotopes

The invention is superior for imaging, because the I-131 has a half-life of 13 days. Thus, assume on day 1, a patient is imaged, and treatment is desired. The physician will be unable to evaluate the progress made for some considerable time with the I131 labeled chlorotoxin, particularly with high dose I-131, because it will take so long for agent to decay that was originally present.

Utilizing I-123, a low dose for imaging is administered, the image taken, and then treatment can occur. Once the half-lives have lowered the radioactivity below the imaging level, then new imaging agent can be administered, and analyzed for uptake. If a patient is progressing, there will be a smaller area or volume of uptake shown and progress can be analyzed more quickly. If another dose of radiotagged chlorotoxin is needed, because the I-123 has a half life which is a fraction (about 7%) of the half-life of I-131, treatment can be repeated many times more rapidly.

The other advantage of the invention is that there is always difficulty in finding drugs that will be effective on brain cancers. Existing literature proposes using a localized pump for intracavitary dosing. Hockaday et al, “Imaging Glioma Extent with 131 I-TM-601,” 46(4) J. Nucl. Med. 580 (Socy. Nucl. Med. 2005).; the invention herein could be administered by IV on a systemic basis, because even if lodged in organs, the half-life is such that the radiopharmaceutical will be harmless fairly quickly.

The method which would be most ideal would be to image the tumor with I-123 chlorotoxin, and then treat the tumor with either I-131 chlorotoxin or I-124 chlorotoxin. The treatment with 1-131 chlorotoxin is suggested (Mamelak et al). No reference or combination of references has been made or suggested to enabling the serial use by combining the lyophilization method described herein and in Kuperus et al (issuing as U.S. Pat. No. 7,229,603, Jun. 12, 2007) and utilizing either I-124 or I-123 in conjunction with a chlorotoxin for imaging, and after that particular imaging, using, in conjunction with a chlorotoxin, I-124 or I-131 as a tumor-targeting agent.

The method of stabilization and lyophilization of the radiopharmaceutical is described as follows as described in U.S. application Ser. No. 10/904,099. The quantities proposed to be used, instead of MIBG as referenced in U.S. application Ser. No. '099, are 1.0 mg of TM-601 available Transmolecular, Inc. of Birmingham, Ala. The amount preferred for imaging is 10-20 millicuries. This invention permits very high doses of I-131, but the preferred dose at time of administration is as selected by the physician. For local administration as a therapeutic for tumor diminution, generally, not more than 100 mCi of 1-131 would be proposed as suggested in Shen et al 71 J. of Neuro-Oncology 113; for IV administration, a lesser dose may be preferred to minimize cell damage during localization to a tumor. Other preferred doses are described in Imaging Glioma Extent with 131-I-TM-601. Other chlorotoxin peptides for radiolabeling are described in Yue et al, “Synthesis, Expression And Purification Of A Type Of Chlorotoxin-Like Peptide From The Scorpion, Buthus Martenssi Darsch, And Its Acute Toxicity Analysis,” 27 Biotechnology Letter 1597-1603 (Springer 2005) and literature cited in that article. See also Zhu et al, “Molecular Characterization Of A New Scorpion Venom Lipolysis Activating Peptide Evidence For Disulfide Bridge-Mediated Functional Switch Of Peptides,” FEBS letters, Science Direct 27 Nov. 2006

As is standard medical practice, potassium iodide may be administered before and after administration to minimize thyroid uptake of iodine.

The preferred labeling method is the Iodo-Gen bead method. Alternatively, by known methods, a stannous compound, buffer and iodide in solution can be fluxed with the TM-601 or other chlorotoxin for labeling and filtered. The invention enables particularly high purity to be obtained and achieved. For places that choose to use facilities on The chlorotoxin can also be used with a DOTA or TETA linker to the protein to connect to other metals not referenced in the literature such as Cu-64, Cu-67, Lu-177, Actitium-225 and Yt-90. Additionally, a boron placed on the protein would be advantageous to enable exterior neutron beam bombardment to cause a heavy particle discharge. Chlorotoxin can be obtained as a synthetic peptide from TransMolecular, Inc., Birmingham, Ala. and reconstituted into a volume of 0.5 ml with sterile sodium phosphate, pH 7.6 at a concentration of 1 mg/ml. Shen S. et al, “Radiation dosimetry of 131 I-chlorotoxin for targeted radiotherapy in glioma-bearing mice,” 71 J. of Neuro-Oncology 113-119 (Springer 2005). The radiolabeling is described in Shen, S, just cited Table 3 of that article contains recommended doses for humans.

In contrast to the Wolfangel '556 invention which stated: “the lyophilization step itself generally takes about 24 hours to perform,” the present invention proposes to produce a stable radiopharmaceutical complex by a lyophilization process which “freeze-dries” the complex in five hours or less, normally 2-4 hours, and then requires no further refrigeration. A recent text by Saha on Nuclear Pharmacology refers to the necessity of a heating step; this invention eliminates that time-consuming and potentially deleterious step.

The preferred mode of illustrating the stabilization and lyophilization aspects of the invention, particularly for diagnostics is showing the use in conjunction with Iodine-123 (“I-123” (123 being the sum of the protons and neutrons)) radionuclides. The following illustrates the compositions and processes of the stabilization and lyophilization part of this invention, but is not meant to limit the scope of the invention in any way. The illustration is that an I-123 labeled compound such as meta-iodo-benzyl-guanidine (“MIBG”) is prepared. The concentration is increased so that ultimately one-half milliliter or less will equal one dose. For example the usual dose of I-123 MIBG for a typical patient would be 10 mCi (millicuries). Because the half life is 12 hours, in order to allow for normal radioactive decay in shipment so that the dose is 10 mCi upon administration, 36 mCi would be mixed on the prior day anticipating overnight shipment. This would apply for a radioiodonated chlorotoxin as well.

With respect to the equipment, the condensing system is heavily insulated.

A hose runs from the top or side of the stainless steel pot of the primary condenser to the vacuum pump.

A vacuum pump capable of producing a vacuum of at least 10-4 Torr would be used to evacuate the chamber. An appropriate vacuum pump is model RV-12 available from BOCEdwards, an international company, of Wilmington, Mass., which can be contacted through the internet.

In order to achieve the composition contemplated in this invention, the primary condensing coil is readied at or below −40 deg. C. Promptly after mixing the radiopharmaceutical composition, the vial containing the radiopharmaceutical composition, in the preferred mode the 0.36 ml. of aqueous I-123MIBG, is stoppered with the lyophilization stopper, with the lyophilization stopper in a position to permit passage of vapour. The vial and stopper will be fully sealed at the end of the process. The vial(s) is (are) placed into the tray and a sufficient amount of liquid nitrogen is poured onto the tray in order to flash freeze the vials by the heat transfer from the aqueous I-123MIBG through the sides of the vial. Because of the small quantity which is used and the high surface area of the vial, the freezing occurs virtually instantaneously. The tray is placed into a stoppering frame in the chamber with the inner tube connected and installed so that at the end of the procedure, before the vacuum is broken, the port to the inner tube can be opened and the tube will inflate and force the stoppers fully into the vials in order to seal them.

As the liquid nitrogen evaporates off, a thermistor on one of the vials is connected to the electrical connector on the rubber stopper which connects to an outside temperature monitoring device. The liquid nitrogen is allowed to evaporate, all the while maintaining the temperature of the vial at or below −10 degrees C.

The top of the chamber is installed and forms a seal with the cylindrical side of the chamber. After evaporation of the liquid nitrogen, the gas valve on top of the chamber is closed, and the rubber stopper is installed.

After the tray containing the flash-frozen vials is placed into the chamber, and the chamber has been sealed, the vacuum pump is turned on. A vacuum pressure is first felt in the primary condenser and any vapor in the chamber begins to flow out through the secondary condenser and freezes in the primary condenser which is kept at a temperature above the boiling point of oxygen, meaning preferably kept at about −40 degrees C. A reasonably skilled practitioner in the art would recognize that at 10-2 Torr and −40 degree C. the amount of oxygen present would be sufficiently low that the danger of oxygen oxidation damage from liquid oxygen if the temperature is lowered below −40 degree C. is eliminated. The preferable level for activating the secondary condenser is 10-3 Torr but 10-2 Torr is appropriate. When the vacuum pump gauge shows the preferred level of 10-3 Torr, usually after about 20 minutes, liquid Nitrogen at −196 degrees C. is allowed to flow through the secondary condenser and cool the stainless steel tube contained in the secondary condenser through which gas evacuated from the chamber is flowing. The very cold liquid Nitrogen in the secondary compressor is used to increase the temperature difference between the secondary condenser and the vial contents to accelerate the lyophilization. The secondary condenser is placed in series with the primary condenser and the evacuated chamber containing the tray of vials. The secondary condenser takes over as the larger and faster heat sink to capture the vaporized water. A reasonably skilled practitioner would understand that the vacuum pump continues to run to the end of the procedure, and the pressure continues to drop to the rated capacity of the vacuum pump. A reasonably skilled practitioner would know that the pump referenced, the model RV-12 available from BOCEdwards, has a rated capacity of approximately 10-6. Thus, after the system has been sealed and the pump is turned on, the pressure drops through the 10-2 Torr and 10-3 Torr levels to the rated capacity of the vacuum pump.

Because the acrylic chamber has no refrigeration, the temperature of the vial and the vial contents tend to rise above 0 degrees C. after all of the water is removed. This signals the completion of the cycle. The thermistor probe connected through the rubber stopper to the outside monitoring device enables the monitoring of the vial temperature. The vials would then be sealed in partial pressure of pharmaceutically inert gas that is fully dehydrated or “dry,” meaning gas that is non-reactive with the pharmaceutical composition, the gas preferably being argon or nitrogen. An inner tube will have been placed in the chamber to be inflated to force the stoppers into the vial to seal them. An auxiliary cylinder of gas that is chemically inert relative to the lyophilized radionuclide is used to gradually inflate the inner tube through the valve to force the stoppers into the vials. The vacuum is broken. The vial stoppers further secured with an aluminum seal. At the end of the process upon warming, the water which was frozen and subsequently melted will be drained from the primary condenser.

The vials are ready to be shipped with predictable half lives for the radionuclide and a stabilized ligand in powdered form.

If it is desired to accelerate the lyophilization process, inert gas may be admitted through the gas valve into the chamber to displace any oxygen and enable the secondary condenser to be turned on sooner. The displacement is necessary to prevent accumulation of liquid oxygen in the secondary condenser. In the ordinary procedure, if the secondary condenser is activated before the 10-3 level is reached, there is a risk of collecting liquid oxygen which is potentially explosive.

The secondary condenser is in series with the primary condenser, and could be located subsequent to the primary condenser in the evacuation and condensing system. The speed of the lyophilization process is positively influenced by the lowering of the vapor pressure external to the material being dried. Secondly, the speed is positively influenced by the greater temperature difference between product being cooled and the temperature of the condenser where the water is being collected.

The radioactive diagnostic radiopharmaceutical in this invention requires no further cold or refrigerated storage, including with respect to shipping, subsequent to stabilization. The lyophilized radiopharmaceutical composition is reconstituted “on site” for administration to patients by the addition of a suitable diluent to bring the radiopharmaceutical complex into solution at the time of administration to the patient. For administration, the I-123 labeled MIBG in the vial must be reconstituted. Because of the minute quantity of material, the vial of radionuclide complex, in the preferred mode the I-123 labeled MIBG will appear empty. The MIBG ligand is stable for several days because of the absence of water which is the primary substance from which free radicals are generated by gamma ray collisions with water molecules. The gamma rays are being emitted by the radionuclide, that is the I-123. The health care provider would add up to 2 ml. of sterile normal saline. The desired dose would be withdrawn and measured in a dose calibrator of a type manufactured by Capintec of Montville, N.J. If the glass vial is measured in the dose calibrator, the person measuring the dose must recognize that the glass vial will decrease the apparent activity. Upon calibration of the desired dose, the I-123 MIBG now re-dissolved in the solution is promptly administered to the patient. The advantages are that the flash freezing and lowering of vapor pressure result in quick formation and evaporation or sublimation (evaporation from ice to water vapour (a gas)) of water from the I-123 MIBG. The I-123 MIBG need not be shipped frozen in dry ice nor need it be shipped for overnight delivery. Shipping in dry ice over a weekend is generally not commercially practical. The I-123 MIBG can be shipped over the weekend and be used on Monday while simply maintaining it at room temperature or below. In order to establish the advantages of the novel process and resulting composition, a series of tests were run utilizing meta iodo benzyl guanidine (MIBG) in which the radionuclide I-131 was the iodine in the MIBG.

The efficacy of the invention for iodinated compounds is demonstrated as follows:

The MIBG was prepared as follows: eight vials were prepared of MIBG in solution with a radioactive concentration of MIBG of 1 mCi per vial. The MIBG in six of those vials were then stabilized and lyophilized according to the process described in this invention. One vial was frozen and maintained at a temperature of −10 degrees, and another vial was simply refrigerated at approximately 5 degrees.

Six vials were prepared according to the process in this invention in order to enable several to be reconstituted from the lyophilized state and their activity tallied.

The radioactive concentration of MIBG per vial was 1 mCi per vial.

The results showing the percent of iodine remaining bound to the MIBG are set forth in table I. One each of the vials was reconstituted after 24, 48, 72 and 168 hours respectively.

| | | | | | 168 hours |
|---|---|---|---|---|---|
| | 0 hours | 24 hours | 48 hours | 72 hours | (1 wk.) |
| Lyophilized | 96.3% | 97% | 96.6% | 96.2% | 95.9% |
| and stabilized | | | | | |
| per invention | | | | | |
| stored at | | | | | |
| room temp. | | | | | |
| Frozen −10° | 96.3% | 94% | 91% | 84% | 72% |
| Refriger- | 96.3% | 92% | 85% | 77% | 55% |
| ated ~+5° | | | | | |

In sum, the radiolysis damage was virtually eliminated from the composition stabilized and lyophilized under this invention while, as the prior art suggests, MIBG that was not so stabilized and lyophilized per this invention deteriorated sharply in activity. As another example, 1-131 Hippuran was prepared. The 1-131 Hippuran was prepared as follows: 9 vials were prepared of 1-131 Hippuran in solution with a radioactive concentration of MIBG of 1 mCi per vial. Each vial had 4 cc. The 1-131 in seven of those vials was then stabilized and lyophilized according to the process described in this invention. One vial was frozen and maintained at a temperature of −10 degrees, and another vial was maintained room temperature. Room temperature was selected because Hippuran is thought to be stable at room temperature even in conjunction with a radioisotope.

The results showing the percent of Hippuran remaining bound to the I-123 are set forth in table 2. One each of the vials was reconstituted after 24, 48, 72 and 168 hours respectively.

TABLE 2

| | 0 hours | 24 hrs | 48 hrs | 72 hrs | 96 hrs | 120 hrs |
|---|---|---|---|---|---|---|
| Lyophilized | 98% | 98.4% | 98.6% | 98% | 98.4% | 98.5% |
| and stabilized | | | | | | |
| per invention | | | | | | |
| stored at | | | | | | |
| room temp. | | | | | | |
| Frozen −10° | 98% | 97.8% | 97% | 94% | 92.5% | 91 |
| Room Temp. | 98% | 96% | 95% | 94.5% | 92% | 90% |

In sum, the radiolysis damage was virtually eliminated from the composition stabilized and lyophilized under this invention.

If one desires to ship product, maintaining a product reliably frozen even at −10 degrees is difficult and expensive as a practical matter; this invention makes such shipment practical over the techniques of the prior art. One reference has suggested that storage at −70° C. can limit autoradiolysis damage, but even in that article, the percent free iodine, e.g. unbonded iodine, rose from what appears to be 1.6% to 4.3% in 24 hours. Wahl, Inhibition of Autoradiolysis of Radiolabeled monoclonal Antibodies by Cryopreservation, 31(1) J. Nucl. Med. 84-89 (January 1990). Conversely, putting those results in a form analogous to Table I, the percentage of free iodine in the Wahl article commenced at 98.4% and fell in 24 hours to 95.7% in Wahl's Table 1. The contrast between that fall in bonded iodine in 24 hours of some 3.7% in the Wahl reference versus a fall of 0.4% during a week for the composition stabilized and lyophilized per this invention illustrates the sharp advantage of the present method and resulting composition. In addition, it is not practical in real-world conditions to replenish the cooling fluid to maintain −70° C. much less to ship it cost-effectively.

The micro quantities involved for radionuclide complexes such as I-123 MIBG substantially reduce the exposure of production workers and health care providers because minute quantities are involved.

More generally, the preferred mode will use compounds that have a half life of one hour to a maximum of 12 hours. Longer half lives are less used because of slower radioactive decay exposing the body to increased radiation. It is generally preferable to apply the flash-freezing first because application of the reduced pressure may cause the solution to boil out of the vial.

Applying the invention more generally, the intent is to utilize the invention to produce stabilized radiopharmaceutical compositions. Such stabilized radiopharmaceutical compositions include radionuclides which are combined with ligand useful for diagnosis or diagnostic treatment or therapy to form radiopharmaceutical complexes in solution or suspension. These complexes then are lyophilized in accord with the above procedure according to the desired radioactivity level for the selected radionuclide. The form of radiopharmaceutical composition lyophilized according to this invention can be stored until needed for use. This invention allows for the central preparation, purification and shipment of a stabilized form of a radiopharmaceutical complex which merely is reconstituted prior to use. Thus, complicated or tedious formulation procedures, as well as unnecessary risk of exposure to radiation, at the site of use are avoided.

The radioactive diagnostic radiopharmaceutical in this invention requires no further cold or refrigerated storage, including with respect to shipping, subsequent to stabilization.

The invention is not meant to be limited to the disclosures, including best mode of invention herein, and contemplates all equivalents to the invention and similar embodiments to the invention for humans, mammals and plant science. Equivalents include combinations with or without stabilizing agents and adjuncts that assist in reservation, and their pharmacologically active racemic mixtures, diastereomers and enantiomers and their pharmacologically acceptable salts in combination with suitable pharmaceutical carriers.

We claim:

1. A stabilized radiolabeled chlorotoxin agent for imaging comprising:
a radioisotope selected from the group of iodine isotopes;
a chlorotoxin selected from the venom selected from the group of scorpions having venom with chlorotoxin;
radioiodating said chlorotoxin agent to create a radiopharmaceutical composition;
upon such radioiodination, evacuating a sealable chamber containing a flash frozen amount of said radiopharmaceutical composition in at least one lyophilization-stoppered but as yet unsealed vial;
said evacuating of said sealable chamber occurring by a vacuum pump connected by an evacuation tube passing through a primary condenser and a secondary condenser to lower pressure to below 10⁻² Torr which is sufficient to eliminate the explosive potential of liquid oxygen while maintaining the temperature of said primary condenser above the boiling point of oxygen at said pressure;
activating said secondary condenser while said pump continues to operate, to reduce said evacuation tube temperature below the boiling point of oxygen in order to accelerate the removal of water from said sealable chamber, thereby reducing more rapidly the presence of water molecules, including radiolysis degenerated water molecules, and reducing attendant free radical damage to said radiopharmaceutical composition, and increasing the predictability of the integrity of the radiopharmaceutical composition; and
turning off said vacuum pump and restoring the ambient pressure in the sealable chamber to approximately atmospheric pressure with a pharmaceutically inert gas upon completion of the desired removal of water; and
sealing the said at least one vial in order to preclude entry of external fluid,
thereby creating a stable, lyophilized storable imaging agent.

2. The agent according to claim 1, further comprising:
said radioisotope being selected from the group of Iodine-123 or Iodine 124.

3. The agent according to claim 2, further comprising:
said radioisotope being Iodine-123.

4. The chlorotoxin according to claims 1, 2, or 3, further comprising:
said chlorotoxin being TM-601.

5. A stabilized radiolabeled chlorotoxin agent for therapeutic administration comprising:
a radioisotope selected from the group of iodine isotopes;
a chlorotoxin selected from the venom selected from the group of scorpions having venom with chlorotoxin;
radioiodating said chlorotoxin agent to create a radiopharmaceutical composition;
upon such radioiodination, evacuating a sealable chamber containing a flash frozen amount of said radiopharmaceutical composition in at least one lyophilization-stoppered but as yet unsealed vial;
said evacuating of said sealable chamber occurring by a vacuum pump connected by an evacuation tube passing through a primary condenser and a secondary condenser to lower pressure to below 10⁻² Torr which is sufficient to eliminate the explosive potential of liquid oxygen while maintaining the temperature of said primary condenser above the boiling point of oxygen at said pressure;
activating said secondary condenser while said pump continues to operate, to reduce said evacuation tube temperature below the boiling point of oxygen in order to accelerate the removal of water from said sealable chamber, thereby reducing more rapidly the presence of water molecules, including radiolysis degenerated water molecules, and reducing attendant free radical damage to said radiopharmaceutical composition, and increasing the predictability of the integrity of the radiopharmaceutical composition; and
turning off said vacuum pump and restoring the ambient pressure in the sealable chamber to approximately atmospheric pressure with a pharmaceutically inert gas upon completion of the desired removal of water; and
sealing the said at least one vial in order to preclude entry of external fluid,
thereby creating a stable, lyophilized storable imaging agent.

6. The agent according to claim 5, further comprising:
said radioisotope being selected from the group of Iodine-124 or Iodine 131.

7. The agent according to claim 6, further comprising:
said radioisotope being Iodine-131.

8. The agent according to claims 5, 6, or 7, further comprising:
said chlorotoxin being TM-601™.

9. A stabilized radiolabeled chlorotoxin agent for imaging comprising:
a metal radioisotope selected from the group of metal isotopes of Cu-64, At-225, Cu-67, Yt-90 or Lu-177;
a chlorotoxin selected from the venom selected from the group of scorpions having venom with chlorotoxin;
a cross-linking compound selected form the group of DOTA or TETA to link said chlorotoxin to said metal radioisotope;
radiolabeling said chlorotoxin agent to create a radiopharmaceutical composition;
upon such radioiodination, evacuating a sealable chamber containing a flash frozen amount of said radiopharmaceutical composition in at least one lyophilization-stoppered but as yet unsealed vial;
said evacuating of said sealable chamber occurring by a vacuum pump connected by an evacuation tube passing through a primary condenser and a secondary condenser to lower pressure to below 10⁻² Torr which is sufficient to eliminate the explosive potential of liquid oxygen while maintaining the temperature of said primary condenser above the boiling point of oxygen at said pressure;
activating said secondary condenser while said pump continues to operate, to reduce said evacuation tube temperature below the boiling point of oxygen in order to accelerate the removal of water from said sealable chamber, thereby reducing more rapidly the presence of water molecules, including radiolysis degenerated water molecules, and reducing attendant free radical damage to said radiopharmaceutical composition, and increasing the predictability of the integrity of the radiopharmaceutical composition; and
turning off said vacuum pump and restoring the ambient pressure in the sealable chamber to approximately atmospheric pressure with a pharmaceutically inert gas upon completion of the desired removal of water; and
sealing the said at least one vial in order to preclude entry of external fluid,
thereby creating a stable, lyophilized storable imaging agent.

10. The agent according to claim 9, further comprising:
said chlorotoxin being TM-601™.

11. The agent according to claim 9, further comprising:
said chlorotoxin being derived from venom of scorpion Buthus martensii Karsch.

12. The agent according to claim 9, further comprising:
said chlorotoxin being derived from venom of scorpion Leiurus quinquestriatus.

13. A method of treatment of a patient having a malignant tumor comprising the following steps:
preparing a stabilized radiolabeled chlorotoxin agent having a chlorotoxin selected from the venom selected from the group of scorpions having venom with chlorotoxin and being labeled with an I-123 radioisotope;
said preparing of said stabilized I-123 radiolabeled chlorotoxin agent occurring by evacuating a sealable chamber containing a flash frozen amount of said I-123 agent in at least one lyophilization-stoppered but as yet unsealed vial;
said evacuating of said sealable chamber occurring by a vacuum pump connected by an evacuation tube passing through a primary condenser and a secondary condenser to lower pressure to below 10⁻² Torr which is sufficient to eliminate the explosive potential of liquid oxygen while maintaining the temperature of said primary condenser above the boiling point of oxygen at said pressure;
activating said secondary condenser while said pump continues to operate, to reduce said evacuation tube temperature below the boiling point of oxygen in order to accelerate the removal of water from said sealable chamber, thereby reducing more rapidly the presence of water molecules, including radiolysis degenerated water molecules, and reducing attendant free radical damage to said radiopharmaceutical composition, and increasing the predictability of the integrity of the radiopharmaceutical composition; and
turning off said vacuum pump and restoring the ambient pressure in the sealable chamber to approximately atmospheric pressure with a pharmaceutically inert gas upon completion of the desired removal of water; and
sealing the said at least one vial in order to preclude entry of external fluid;
preparing a stabilized radiolabeled chlorotoxin agent having a chlorotoxin selected from the venom selected from the group of scorpions having venom with chlorotoxin and being labeled with an iodine radioisotope selected from the group of I-124 or I-131;
said preparing of said stabilized iodine isotope radiolabeled chlorotoxin agent occurring by evacuating a sealable chamber containing a flash frozen amount of said iodine radiolabeled chlorotoxin agent in at least one lyophilization-stoppered but as yet unsealed vial;
said evacuating of said sealable chamber occurring by a vacuum pump connected by an evacuation tube passing through a primary condenser and a secondary condenser to lower pressure to below 10⁻² Torr which is sufficient to eliminate the explosive potential of liquid oxygen while maintaining the temperature of said primary condenser above the boiling point of oxygen at said pressure;
activating said secondary condenser while said pump continues to operate, to reduce said evacuation tube temperature below the boiling point of oxygen in order to accelerate the removal of water from said sealable chamber, thereby reducing more rapidly the presence of water molecules, including radiolysis degenerated water molecules, and reducing attendant free radical damage to said radiopharmaceutical composition, and increasing the predictability of the integrity of the radiopharmaceutical composition; and
turning off said vacuum pump and restoring the ambient pressure in the sealable chamber to approximately atmospheric pressure with a pharmaceutically inert gas upon completion of the desired removal of water; and
sealing the said at least one vial in order to preclude entry of external fluid;
reconstituting said stabilized I-123 agent and performing a diagnostic scan on a patient;
reconstituting said stabilized iodine-131 radiolabeled chlorotoxin agent and performing a therapeutic administration of said stabilized iodine-131 radiolabeled chlorotoxin agent on a patient;
continuing said performing said diagnostic scan and said performing said therapeutic administration until said tumor is reduced in size.

14. The method according to claim 13, further comprising:
said chlorotoxin agent being TM-601™.

15. The method according to claim 13, further comprising:
said chlorotoxin being derived from venom of scorpion Buthus martensii Karsch.

16. The method according to claim 13, further comprising:
said chlorotoxin being derived from venom of scorpion Leiurus quinquestriatus.
